(19) United States
(12) Patent Application Publication
(10) Pub. No.: US 2024/0193781 A1
(43) Pub. Date: Jun. 13, 2024

(54) CONTRAST STATE DETERMINATION DEVICE, CONTRAST STATE DETERMINATION METHOD, AND PROGRAM

(71) Applicant: FUJIFILM Corporation, Tokyo (JP)

(72) Inventor: Keita OTANI, Tokyo (JP)

(73) Assignee: FUJIFILM Corporation, Tokyo (JP)

(21) Appl. No.: 18/587,960

(22) Filed: Feb. 27, 2024

Related U.S. Application Data
(63) Continuation of application No. PCT/JP2022/025287, filed on Jun. 24, 2022.

(30) Foreign Application Priority Data
Aug. 31, 2021 (JP) 2021-141457

(51) Int. Cl. G06T 7/00

(52) U.S. Cl. CPC G06T 7/0016; G06T 2200/04; G06T 2207/10081; G06T 2207/20081; G06T 2207/30056; G06T 2207/30084

(57) ABSTRACT

Provided are a contrast state determination device, a contrast state determination method, and a program that quickly, accurately, and robustly determine a contrast state even in a case where there is an organ that is not included in an image. A plurality of two-dimensional images including information of slice images of a subject at different positions are acquired from a first image series captured before or after a contrast agent is injected into the subject, an index value related to a contrast state is estimated from each of the plurality of two-dimensional images, and the contrast state of the first image series is determined on the basis of each of a plurality of the index values.

### CROSS-REFERENCE TO RELATED APPLICATIONS

The present application is a Continuation of PCT International Application No. PCT/JP2022/025287 filed on Jun. 24, 2022 claiming priority under 35 U.S.C. § 119(a) to Japanese Patent Application No. 2021-141457 filed on Aug. 31, 2021. Each of the above applications is hereby expressly incorporated by reference, in its entirety, into the present application.

### BACKGROUND OF THE INVENTION

### 1. Field of the Invention

The present invention relates to a contrast state determination device, a contrast state determination method, and a program that determine a contrast state from an image captured by contrast imaging, and particularly relates to a technique that determines a contrast state from a two-dimensional image.

### 2. Description of the Related Art

The appearance of a dynamic contrast-enhanced computed tomography (CT) image captured by a CT examination using a contrast agent differs significantly depending on a contrast time phase, and accurate understanding of the contrast time phase is required for a process depending on contrast such as blood vessel extraction. Contrast information may be included in a digital imaging and communications in medicine (DICOM) tag. However, the contrast information is not necessarily included in the DICOM tag and may be included therein by mistake. Therefore, it is required to understand the contrast time phase from the image.

JP5357818B discloses a technique that determines the presence or absence of contrast of a liver. In addition, the contrast includes a plurality of phases (for example, pre-contrast phase, an arterial phase, a portal phase, and an equilibrium phase). Automatic Contrast Phase Estimation in CT Volumes, Michal Sofka, Dijia Wu, Michael S\ddot{ } uhling, David Liu, Christian Tietjen, Grzegorz and Soza, S. Kevin Zhou also <URL:https://link.springer.com/content/pdf/10.1007%2F978-3-642-23626-6_21.pdf> discloses a technique that automatically determines these phases from an image.

### SUMMARY OF THE INVENTION

Information of the contrast time phase is required for another process that depends on the contrast time phase in the latter stage, and a high-speed operation is required. However, the technique disclosed in Automatic Contrast Phase Estimation in CT Volumes, Michal Sofka, Dijia Wu, Michael S\ddot{ } uhling, David Liu, Christian Tietjen, Grzegorz Soza, and S. Kevin Zhou <URL:https://link.springer.com/content/pdf/10.1007%2F978-3-642-23626-6_21.pdf> has a problem that it takes time because a three-dimensional (3D) contrast image is processed without any change. Further, the technique disclosed in JP5357818B has a problem that it is not possible to determine the contrast time phase in a case where a specific region is not included in the contrast image.

The present invention has been made in view of these circumstances, and an object of the present invention is to provide a contrast state determination device, a contrast state determination method, and a program that quickly, accurately, and robustly determine a contrast state even in a case where there is an organ that is not included in an image.

In order to achieve the object, according to an aspect of the present invention, there is provided a contrast state determination device comprising: at least one processor; and at least one memory that stores commands to be executed by the at least one processor. The at least one processor acquires a plurality of two-dimensional images including information of slice images of a subject at different positions from a first image series captured before or after a contrast agent is injected into the subject, estimates an index value related to a contrast state from each of the plurality of two-dimensional images, and determines the contrast state of the first image series on the basis of each of a plurality of the estimated index values.

That is, the contrast state determination device acquires at least a first two-dimensional image including information of a first slice image of the subject at a first position and a second two-dimensional image including information of a second slice image at a second position different from the first position from the first image series captured before or after the contrast agent is injected into the subject, estimates a first index value related to the contrast state from the first two-dimensional image, estimates a second index value related to the contrast state from the second two-dimensional image, and determines the contrast state of the first image series on the basis of at least the first index value and the second index value. The contrast state determination device can further acquire a third two-dimensional image including information of a third slice image of the subject at a third position different from the first position and the second position from the first image series, estimate a third index value related to the contrast state from the third two-dimensional image, and determine the contrast state of the first image series on the basis of the first to third index values.

According to this aspect, the contrast state of the image series is determined on the basis of each index value estimated from the two-dimensional images of the subject at a plurality of different positions. Therefore, it is possible to quickly, accurately, and robustly determine the contrast state even in a case where there is an organ that is not included in the image.

Preferably, the index value is a certainty of belonging to each of a plurality of the contrast states, and the at least one processor derives an index value obtained by integrating the plurality of index values and determines the contrast state of the first image series on the basis of the integrated index value.

Preferably, the contrast state determination device further comprises a first learning model that, in a case where a two-dimensional image based on an image series captured before or after the contrast agent is injected into the subject is input, outputs a certainty of belonging to each of the plurality of contrast states. Preferably, the at least one processor inputs the plurality of two-dimensional images to the first learning model to estimate the plurality of index values.

Preferably, the index value is an elapsed time from the injection of the contrast agent into the subject, and the at least one processor derives an elapsed time obtained by integrating a plurality of the elapsed times which are the plurality of index values and determines the contrast state of the first image series on the basis of the integrated elapsed time.

Preferably, the index values are an elapsed time from the injection of the contrast agent into the subject and a certainty of the elapsed time, and the at least one processor derives an elapsed time obtained by integrating a plurality of the elapsed times which are the plurality of index values on the basis of a plurality of the certainties which are the plurality of index values and determines the contrast state of the first image series on the basis of the integrated elapsed time.

Preferably, the at least one processor derives the integrated elapsed time on the basis of a product of a plurality of probability distribution models having the elapsed time and the certainty as parameters.

Preferably, the contrast state determination device further comprises a second learning model that, in a case where a two-dimensional image based on an image series captured before or after the contrast agent is injected into the subject is input, outputs the elapsed time from the injection of the contrast agent into the subject and the certainty of the elapsed time. Preferably, the at least one processor inputs the plurality of two-dimensional images to the second learning model to estimate the plurality of elapsed times and the plurality of certainties.

Preferably, the contrast state determination device further comprises a conversion table in which the elapsed time from the injection of the contrast agent into the subject is associated with the contrast state. Preferably, the at least one processor determines the contrast state of the first image series on the basis of the integrated elapsed time and the conversion table.

Preferably, the contrast state determination device further comprises a conversion table in which an elapsed time from the injection of the contrast agent into the subject is associated with the contrast state. Preferably, the at least one processor estimates a plurality of the elapsed times from the injection of the contrast agent into the subject as the plurality of index values, determines a plurality of the contrast states of the first image series on the basis of the plurality of elapsed times and the conversion table, and determines the contrast state of the first image series on the basis of the plurality of contrast states.

Preferably, the two-dimensional image is at least one of the slice image, a maximum intensity projection (MIP) image of a plurality of the slice images, or an average image of the plurality of slice images.

Preferably, the first image series is a three-dimensional image including a liver of the subject, and the contrast state includes at least one of a non-contrast phase, an arterial phase, a portal phase, or an equilibrium phase.

Preferably, the first image series is a three-dimensional image including a kidney of the subject, and the contrast state includes at least one of a non-contrast phase, a corticomedullary phase, a parenchymal phase, or an excretory phase.

In order to achieve the object, according to another aspect of the present invention, there is provided a contrast state determination method comprising: a step of acquiring a plurality of two-dimensional images including information of slice images of a subject at different positions from a first image series captured before or after a contrast agent is injected into the subject; a step of estimating an index value related to a contrast state from each of the plurality of two-dimensional images; and a step of determining the contrast state of the first image series on the basis of each of a plurality of the estimated index values.

One aspect of a program for achieving the above object is a program causing a computer to execute the above-described contrast state determination method. A computer-readable non-transitory storage medium on which the program is recorded may also be included in the present aspect.

According to the present invention, it is possible to quickly, accurately, and robustly determine a contrast state even in a case where there is an organ that is not included in an image.

### BRIEF DESCRIPTION OF THE DRAWINGS

FIG. 1 is a conceptual diagram illustrating an outline of a process by a contrast state determination device according to a first embodiment.

FIG. 2 is a flowchart illustrating a contrast state determination method by the contrast state determination device.

FIG. 3 is a block diagram schematically illustrating an example of a hardware configuration of the contrast state determination device according to the first embodiment.

FIG. 4 is a functional block diagram illustrating an outline of processing functions of the contrast state determination device according to the first embodiment.

FIG. 5 is a conceptual diagram illustrating an outline of a process by a contrast state determination device according to a second embodiment.

FIG. 6 is a functional block diagram illustrating an outline of processing functions of the contrast state determination device according to the second embodiment.

FIG. 7 is a conceptual diagram illustrating an outline of a process by a contrast state determination device according to a third embodiment.

FIG. 8 is a diagram illustrating an example of a process of a number-of-seconds distribution estimation unit.

FIG. 9 is a graph of a function used for variable conversion of Expression (2).

FIG. 10 is a graph of a number-of-seconds distribution that is estimated by parameters estimated by the number-of-seconds distribution estimation unit.

FIG. 11 is a diagram illustrating an example of processes of an integration unit and a maximum point specification unit.

FIG. 12 is a diagram schematically illustrating an example of a machine learning method for generating a regression model applied to the number-of-seconds distribution estimation unit.

FIG. 13 is a diagram illustrating a loss function used during training.

FIG. 14 is a functional block diagram illustrating an outline of processing functions of the contrast state determination device according to the third embodiment.

FIG. 15 is a diagram illustrating an example of a process of a number-of-seconds distribution estimation unit of a contrast state determination device according to a fourth embodiment.

FIG. 16 is a graph of a number-of-seconds distribution that is estimated from parameters estimated by the number-of-seconds distribution estimation unit.

FIG. 17 is a diagram illustrating an example of processes of an integration unit and a maximum point specification unit of a contrast state determination device according to the fourth embodiment.

FIG. 18 is a diagram schematically illustrating an example of a machine learning method for generating a regression model applied to the number-of-seconds distribution estimation unit according to the fourth embodiment.

FIG. 19 is a diagram illustrating Modification Example 1 of data used for input to the contrast state determination device.

FIG. 20 is a diagram illustrating Modification Example 2 of the data used for input to the contrast state determination device.

FIG. 21 is a block diagram illustrating an example of a configuration of a medical information system to which the contrast state determination device is applied.

### DESCRIPTION OF THE PREFERRED EMBODIMENTS

Hereinafter, preferred embodiments of the present invention will be described in detail with reference to the accompanying drawings.

### <<Outline of Contrast State Determination Device 10 According to First Embodiment>>

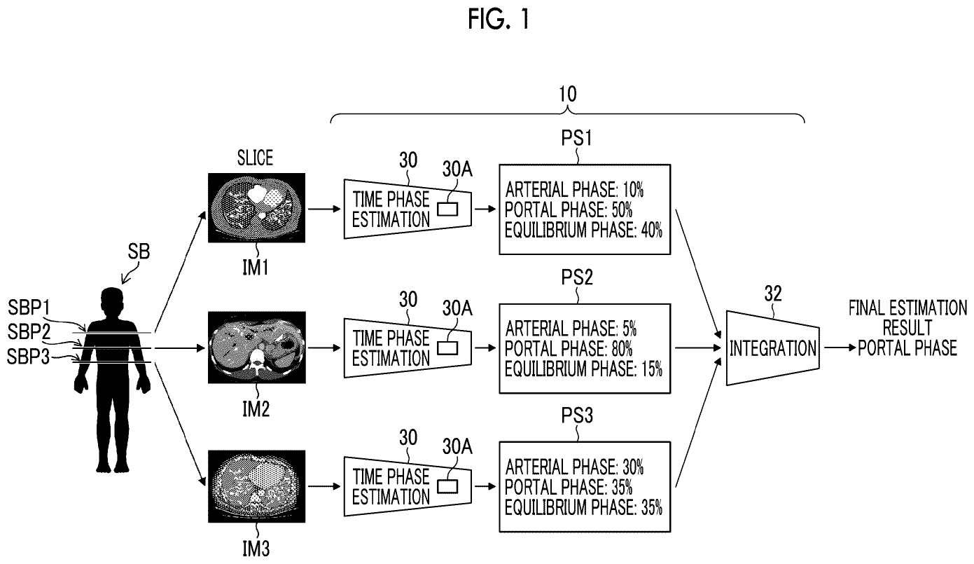

FIG. 1 is a conceptual diagram illustrating an outline of a process by a contrast state determination device 10 according to a first embodiment. Here, an example of the contrast state determination device 10 that estimates a contrast state (contrast time phase) of an image series on the basis of a plurality of slice images of the same image series subjected to contrast imaging using a computed tomography (CT) apparatus will be described. In the first embodiment, the contrast state is estimated by image analysis, using a plurality of slice images in the same series as an input. The term “by image analysis” means by a process based on pixel values constituting image data.

In addition, the contrast state of liver contrast imaging includes at least one of a non-contrast phase, an arterial phase (early arterial phase/late arterial phase), a portal phase, and an equilibrium phase. Further, the contrast state of kidney contrast imaging includes at least one of a non-contrast phase, a corticomedullary phase, a parenchymal phase, or an excretory phase. Here, an example in which the contrast state of an image series obtained by liver contrast imaging is determined will be described.

The contrast state determination device 10 can be implemented using hardware and software of a computer. As illustrated in FIG. 1, the contrast state determination device 10 includes a time phase estimation unit 30 and an integration unit 32.

A plurality of slice images are input to the contrast state determination device 10. The plurality of slice images are images sampled at equal intervals from three-dimensional CT data of the same image series captured before or after the injection of a contrast agent into a patient SB (an example of a “subject”). In the example illustrated in FIG. 1, three images IM1, IM2, and IM3 (an example of “a plurality of slice images” and an example of “a plurality of two-dimensional images”) of the patient SB at different positions SBP1, SBP2, and SBP3 are input to the contrast state determination device 10. In addition, the slice image may be paraphrased as a tomographic image. The slice image may be substantially understood as a two-dimensional image (cross-sectional image).

The time phase estimation unit 30 estimates an index value (an example of an “index value related to a contrast state”) which is a certainty that the image series of the input images will belong to each of a plurality of contrast time phases. A numerical range of the certainty of belonging to cach contrast time phase output from the time phase estimation unit 30 may be “0% to 100%”. In FIG. 1, three time phase estimation units 30 are illustrated in order to illustrate a flow of a process in a case where three different images IM1, IM2, and IM3 are input. However, the time phase estimation units 30 to which each of the images IM1 to IM3 is input are the same (single) processing unit.

The time phase estimation unit 30 includes a trained model 30A (an example of a “first learning model”) that has been trained by machine learning. The trained model 30A is a multi-class classification model that, in a case where two-dimensional images based on a three-dimensional image obtained by contrast imaging are input, outputs the certainty that the image series of the input two-dimensional images belongs to each contrast time phase among a plurality of contrast time phases. For example, the trained model 30A is configured using, for example, a convolutional neural network (CNN). The trained model 30A is trained by a well-known method, using a pair of two-dimensional images based on an image series captured before or after the injection of the contrast agent into the subject and the contrast time phase of the image series as learning data. The time phase estimation unit 30 inputs the input two-dimensional images to the trained model 30A and estimates the index value of each image.

In the example illustrated in FIG. 1, the time phase estimation unit 30 outputs, as the index value of the image IM1, an estimation result PS1 in which the certainty of belonging to the arterial phase among a plurality of contrast states is 10%, the certainty of belonging to the portal phase is 50%, and the certainty of belonging to the equilibrium phase is 40%. In addition, the time phase estimation unit 30 outputs, as the index value of the image IM2, an estimation result PS2 in which the certainty of belonging to the arterial phase is 5%, the certainty of belonging to the portal phase is 80%, and the certainty of belonging to the equilibrium phase is 15%. Further, the time phase estimation unit 30 outputs, as the index value of the image IM3, an estimation result PS3 in which the certainty of belonging to the arterial phase is 30%, the certainty of belonging to the portal phase is 35%, and the certainty of belonging to the equilibrium phase is 35%.

The integration unit 32 derives an index value obtained by integrating a plurality of input index values and determines the contrast state of the image series. The integration unit 32 may integrate the plurality of index values by a maximum value, may integrate the plurality of index values by majority, or may integrate the plurality of index values by a probability sum. The index value integrated by the integration unit 32 is output as a final estimation result. In the example illustrated in FIG. 1, the integration unit 32 integrates the estimation result PS1, the estimation result PS2, and the estimation result PS3 and outputs the portal phase as the final estimation result.

### <<Description of Medical Image Used for Input>>

In a digital imaging and communications in medicine (DICOM) standard that defines a format of a medical image and a communication protocol, a series ID is defined in a unit called a study ID which is an identification code (ID) for identifying an examination type.

For example, in the liver contrast imaging of a certain patient, CT imaging is performed a plurality of times (here, four times) in a range including the liver while changing the imaging timing as described below.
[First imaging] Before the injection of the contrast agent
[Second imaging] 35 seconds after the injection of the contrast agent
[Third imaging] 70 seconds after the injection of the contrast agent
[Fourth imaging] 180 seconds after the injection of the contrast agent

Four types of CT data are obtained by the four imaging operations. The term “CT data” referred to here is three-dimensional data that is composed of a plurality of consecutive slice images (tomographic images), and an aggregate of the plurality of slice images constituting the three-dimensional data (a group of consecutive slice images) is referred to as an “image series”. The CT data is an example of a “three-dimensional image” according to the present disclosure.

The same study ID and different series IDs are given to the four types of CT data obtained by a series of imaging operations including the four imaging operations.

For example, “study 1” is given as a study ID for an examination of liver contrast imaging on a specific patient, and a unique ID is given to each series as follows: “series 1” is given as a series ID to CT data obtained by imaging before the injection of the contrast agent; “series 2” is given to CT data obtained by imaging 35 seconds after the injection of the contrast agent; “series 3” is given to CT data obtained by imaging 70 seconds after the injection of the contrast agent; and “series 4” is given to CT data obtained by imaging 180 seconds after the injection of the contrast agent. Therefore, the CT data can be identified by combining the study ID and the series ID. Meanwhile, in some cases, in the actual CT data, the correspondence relationship between the series ID and the imaging timing (elapsed time since the injection of the contrast agent) is not clearly understood.

In addition, since the size of the three-dimensional CT data is large, it may be difficult to perform the process of determining the contrast state using the CT data as input data without any change.

### <<Contrast State Determination Method>>

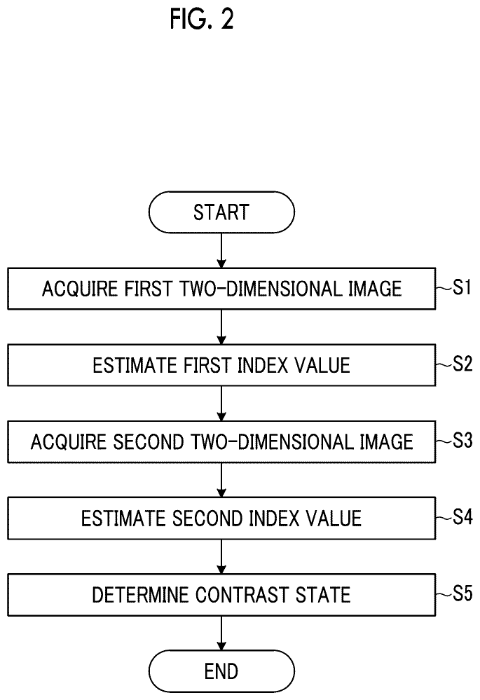

FIG. 2 is a flowchart illustrating each step of a contrast state determination method by the contrast state determination device 10. Here, an example in which the contrast state of an image series obtained by liver contrast imaging is determined will be described.

In Step S1, the contrast state determination device 10 acquires the image IM1 (an example of a “first slice image” and an example of a “first two-dimensional image”) of the patient SB at the position SBP1 (an example of a “first position”) from an image series (an example of a “first image series”) captured before or after the injection of the contrast agent into the patient SB.

In Step S2, the time phase estimation unit 30 inputs the image IM1 acquired in Step S1 to the trained model 30A to estimate a first index value related to the contrast state. Here, the time phase estimation unit 30 calculates, as the first index value, each of the probability of being the non-contrast phase (non-contrast-phase-likeness), the probability of being the arterial phase (arterial-phase-likeness), the probability of being the portal phase (portal-phase-likeness), and the probability of being the equilibrium phase (equilibrium-phase-likeness) and outputs the first index value as the estimation result PS1.

In Step S3, the contrast state determination device 10 acquires the image IM2 (an example of a “second slice image” and an example of a “second two-dimensional image”) of the patient SB at the position SBP2 (an example of a “second position”) from the same image series including the image IM1.

In Step S4, the time phase estimation unit 30 inputs the image IM2 acquired in Step S3 to the trained model 30A to estimate a second index value related to the contrast state. Here, the time phase estimation unit 30 calculates each of the probability of being the non-contrast phase, the probability of being the arterial phase, the probability of being the portal phase, and the probability of being the equilibrium phase as the second index value and outputs the second index value as the estimation result PS2.

Similarly, the contrast state determination device 10 acquires the image IM3, and the time phase estimation unit 30 outputs the estimation result PS3 as the index value related to the contrast state from the image IM3.

In Step S5, the integration unit 32 determines the contrast state of the image series on the basis of the estimation results PS1 to PS3 and outputs the determined contrast state as the final estimation result.

As described above, according to the contrast state determination method by the contrast state determination device 10, the contrast time phase is determined on the basis of a plurality of two-dimensional images. Therefore, accuracy and robustness are higher than those in a case where the contrast time phase is determined on the basis of one two-dimensional image. In addition, inference is faster than that in a case where the contrast time phase is determined on the basis of the three-dimensional image. Further, since the estimation is not performed on the basis of a specific region, the estimation can be performed even in a case where there is an organ that is not included in the image.

Here, the contrast state determination device 10 determines the contrast time phase on the basis of the three images IM1, IM2, and IM3. However, the contrast state determination device 10 may determine the contrast time phase on the basis of two images or may determine the contrast time phase on the basis of four or more images. The process is relatively fast in a case where the number of images is relatively small, and the determination result is relatively highly accurate and robust in a case where the number of images is relatively large.

### <<Example of Hardware Configuration>>

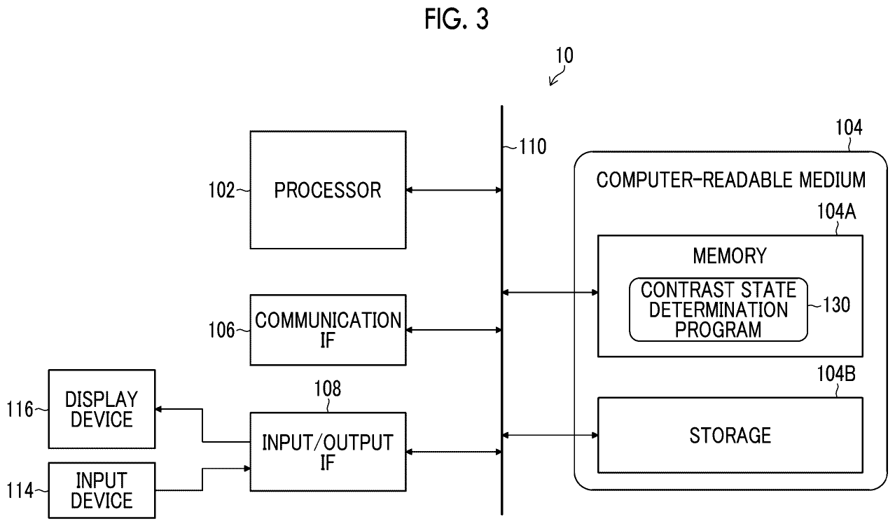

FIG. 3 is a block diagram schematically illustrating an example of a hardware configuration of the contrast state determination device 10 according to the first embodiment. The contrast state determination device 10 can be implemented by a computer system configured using one or a plurality of computers. Here, an example in which one computer executes a program to implement various functions of the contrast state determination device 10 will be described. In addition, the form of the computer that functions as the contrast state determination device 10 is not particularly limited, and the computer may be, for example, a server computer, a workstation, a personal computer, or a tablet terminal.

The contrast state determination device 10 includes a processor 102, a computer-readable medium 104 which is a non-transitory tangible object, a communication interface 106, an input/output interface 108, and a bus 110.

The processor 102 includes a central processing unit (CPU). The processor 102 may include a graphics processing unit (GPU). The processor 102 is connected to the computer-readable medium 104, the communication interface 106, and the input/output interface 108 through the bus 110. The processor 102 reads out various programs, data, and the like stored in the computer-readable medium 104 to execute various types of processing.

The computer-readable medium 104 includes, for example, a memory 104A which is a main storage device and a storage 104B which is an auxiliary storage device. The storage 104B is configured using, for example, a hard disk drive (HDD) device, a solid state drive (SSD) device, an optical disk, a magneto-optical disk, a semiconductor memory, or an appropriate combination thereof. For example, various programs and data are stored in the storage 104B. The computer-readable medium 104 is an example of a “storage device” according to the present disclosure.

The memory 104A is used as a work area of the processor 102 and is used as a storage unit that temporarily stores the program and various types of data read out from the storage 104B. The program stored in the storage 104B is loaded to the memory 104A, and the processor 102 executes commands of the program to function as units for performing various processes defined by the program. The memory 104A stores, for example, a contrast state determination program 130 executed by the processor 102 and various types of data. The contrast state determination program 130 includes the trained model 30A (see FIG. 1) trained by machine learning and causes the processor 102 to perform the processes described with reference to FIGS. 1 and 2.

The communication interface 106 performs a wired or wireless communication process with an external device to exchange information with the external device. The contrast state determination device 10 is connected to a communication line (not illustrated) through the communication interface 106. The communication line may be a local area network or a wide area network. The communication interface 106 can play a role of a data acquisition unit that receives the input of data such as an image.

The contrast state determination device 10 may further include an input device 114 and a display device 116. The input device 114 and the display device 116 are connected to the bus 110 through the input/output interface 108. The input device 114 may be, for example, a keyboard, a mouse, a multi-touch panel, other pointing devices, a voice input device, or an appropriate combination thereof.

The display device 116 is an output interface on which various types of information are displayed. The display device 116 may be, for example, a liquid crystal display, an organic electro-luminescence (OEL) display, a projector, or an appropriate combination thereof.

### <<Functional Configuration of Contrast State Determination Device 10>>

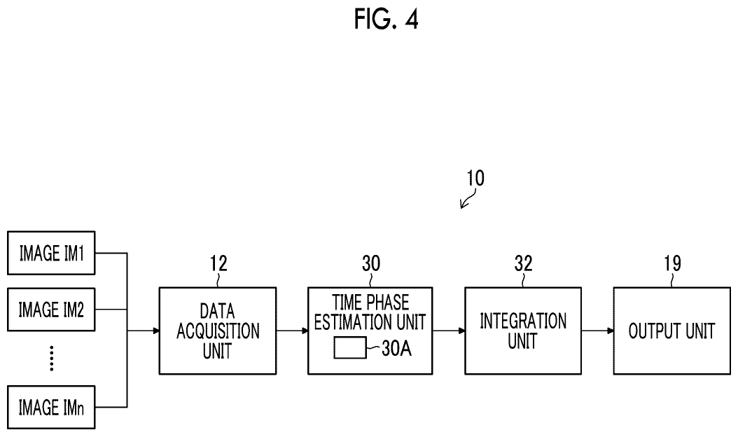

FIG. 4 is a functional block diagram illustrating an outline of processing functions of the contrast state determination device 10 according to the first embodiment. The processor 102 of the contrast state determination device 10 executes the contrast state determination program 130 stored in the memory 104A to function as a data acquisition unit 12, the time phase estimation unit 30, the integration unit 32, and an output unit 19.

The data acquisition unit 12 receives the input of data to be processed. In the example illustrated in FIG. 4, the data acquisition unit 12 acquires images IMi which are slice images sampled from CT data. A subscript i (where i=1 to n) indicates an index number for identifying a plurality of images. FIG. 4 illustrates that n different images can be input. n may be an integer equal to or greater than 2. The data acquisition unit 12 may perform a process of cutting out slice images from the CT data at equal intervals or may acquire the slice images sampled in advance using a processing unit (not illustrated) or the like.

The images IMi acquired through the data acquisition unit 12 are input to the time phase estimation unit 30.

The output unit 19 is an output interface for displaying the contrast state estimated by the integration unit 32 or for providing the contrast state to other processing units. The output unit 19 may include a processing unit that performs, for example, a process of generating data for display and/or a data conversion process for transmission of data to the outside or the like. The contrast time phase estimated by the contrast state determination device 10 may be displayed on a display device (not illustrated) or the like.

The contrast state determination device 10 may be incorporated into a medical image processing device for processing a medical image acquired in a medical institution such as a hospital. In addition, the processing functions of the contrast state determination device 10 may be provided as a cloud service. The method of the contrast state determination process performed by the processor 102 is an example of a “contrast state determination method” according to the present disclosure.

### Second Embodiment

In the first embodiment, the certainty of belonging to each of a plurality of contrast time phases is used as the index value related to the contrast state. However, in a second embodiment, an example will be described in which the elapsed time from the injection of the contrast agent is used as the index value related to the contrast state.

A contrast state determination device 10 according to the second embodiment may have the same hardware configuration as that according the first embodiment. Differences of the second embodiment from the first embodiment will be described.

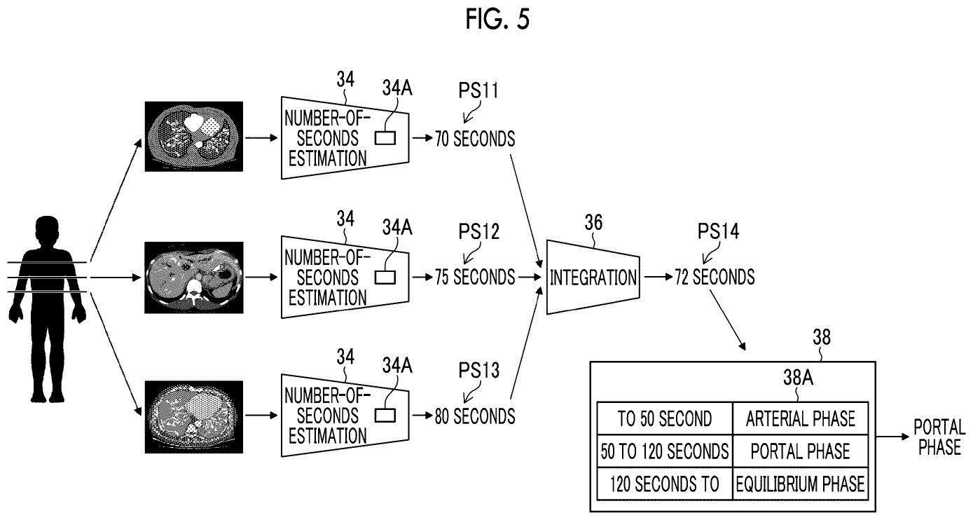

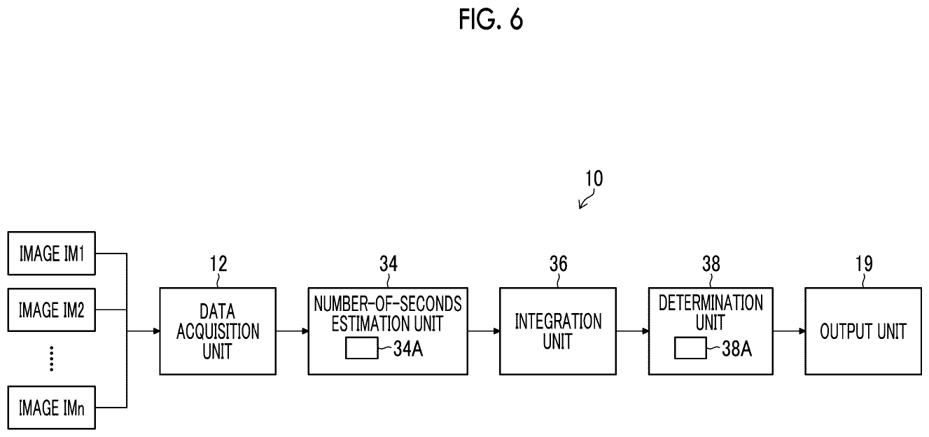

FIG. 5 is a conceptual diagram illustrating an outline of a process by the contrast state determination device 10 according to the second embodiment. In addition, FIG. 6 is a functional block diagram illustrating an outline of processing functions of the contrast state determination device 10 according to the second embodiment.

Here, an example of the contrast state determination device 10 that uses, as an input, a plurality of slice images sampled at equal intervals from three-dimensional CT data of a patient captured by liver contrast imaging using the CT apparatus, estimates the number of seconds of an image series of the plurality of input slice images from the injection of the contrast agent, and determines the contrast state on the basis of the estimated number of seconds will be described. Hereinafter, in the specification, unless otherwise specified, “the number of seconds” includes the meaning of the number of seconds indicating the elapsed time from the injection of the contrast agent.

As illustrated in FIGS. 5 and 6, the contrast state determination device 10 includes a number-of-seconds estimation unit 34 that receives the input of the images IM and estimates the number of seconds of the image series of the images IM as the elapsed time from the injection of the contrast agent, an integration unit 36 that derives an integrated elapsed time from a plurality of input elapsed times, and a determination unit 38 that determines the contrast time phase from the derived elapsed time. The output unit 19 outputs the contrast time phase determined by the determination unit 38 as the final result.

Further, three number-of-seconds estimation units 34 are illustrated in FIG. 5 in order to illustrate a flow of a process in a case where three different images IM are input. However, the number-of-seconds estimation units 34 to which each image IM is input are the same (single) processing unit.

The plurality of slice images input to the contrast state determination device 10 are the same as those in the first embodiment. The number-of-seconds estimation unit 34 estimates the number of seconds of the image series of the input images from the injection of the contrast agent.

The number-of-seconds estimation unit 34 includes a trained model 34A that has been trained by machine learning. In a case where two-dimensional images based on a three-dimensional image obtained by contrast imaging are input, the trained model 34A outputs the number of seconds of the image series of the input two-dimensional images from the injection of the contrast agent. The trained model 34A is configured using, for example, a convolutional neural network. The trained model 34A is trained by a well-known method using, as learning data, a pair of two-dimensional images based on an image series captured before or after the injection of the contrast agent into the subject and the number of seconds of the image series from the injection of the contrast agent. The number-of-seconds estimation unit 34 inputs the input two-dimensional images to the trained model 34A to estimate the elapsed time of the image series of the two-dimensional images from the injection of the contrast agent.

In the example illustrated in FIG. 5, the number-of-seconds estimation unit 34 outputs an estimation result PS11 (an example of a “first elapsed time”) of 70 seconds as the index value of the image IM1, outputs an estimation result PS12 (an example of a “second elapsed time”) of 75 seconds as the index value of the image IM2, and outputs an estimation result PS13 of 80 seconds as the index value of the image IM3.

The integration unit 36 integrates the plurality of input estimation results using any statistical method to estimate the number of seconds of the image series from the injection of the contrast agent. For example, the integration unit 36 may integrate the estimation results by simple averaging, may integrate the estimation results by weighted averaging, or may select any one of the plurality of estimation results and integrate the estimation results. In the example illustrated in FIG. 5, the integration unit 36 integrates the estimation result PS11, the estimation result PS12, and the estimation result PS13, and outputs 72 seconds as a final estimation result PS14.

The determination unit 38 determines the contrast state from the value of the number of seconds integrated by the integration unit 36. The determination unit 38 includes a conversion table 38A. The conversion table 38A may be stored in the computer-readable medium 104. In the conversion table 38A, the elapsed time from the start of the injection of the contrast agent and the contrast time phase are associated with each other. For example, in the case of liver contrast imaging, in the conversion table 38A, less than 50 seconds from the start of the injection of the contrast agent is associated with the arterial phase, 50 seconds or more and less than 120 seconds is associated with the portal phase, and 120 seconds or more is associated with the equilibrium phase. The determination unit 38 determines the contrast state using the conversion table 38A. In the example illustrated in FIG. 5, since the final estimation result PS14 is 72 seconds, the contrast state is determined to be the portal phase.

Here, the integration unit 36 integrates the elapsed times estimated from the images IM1, IM2, and IM3, and the determination unit 38 determines the final contrast state from the integrated elapsed time. However, the number-of-seconds estimation unit 34 may estimate the elapsed time (an example of the first elapsed time”) of the image series of the image IM1, the elapsed time (an example of the “second elapsed time”) of the image series of the image IM2, and the elapsed time of the image series of the image IM3, and the determination unit 38 may determine the contrast state (an example of a “first contrast state”) of the image series of the image IM1, the contrast state (an example of a “second contrast state”) of the image series of the image IM2, and the contrast state of the image series of the image IM3. Then, the integration unit 36 may integrate the determined contrast state of the image series of the image IM1, the determined contrast state of the image series of the image IM2, and the determined contrast state of the image series of the image IM3 to determine the final contrast state.

### Third Embodiment

In a third embodiment, an example will be described in which a probability distribution of the elapsed time from the injection of the contrast agent is used as the index value related to the contrast state. A contrast state determination device 10 according to the third embodiment may have the same hardware configuration as that according to the first embodiment.

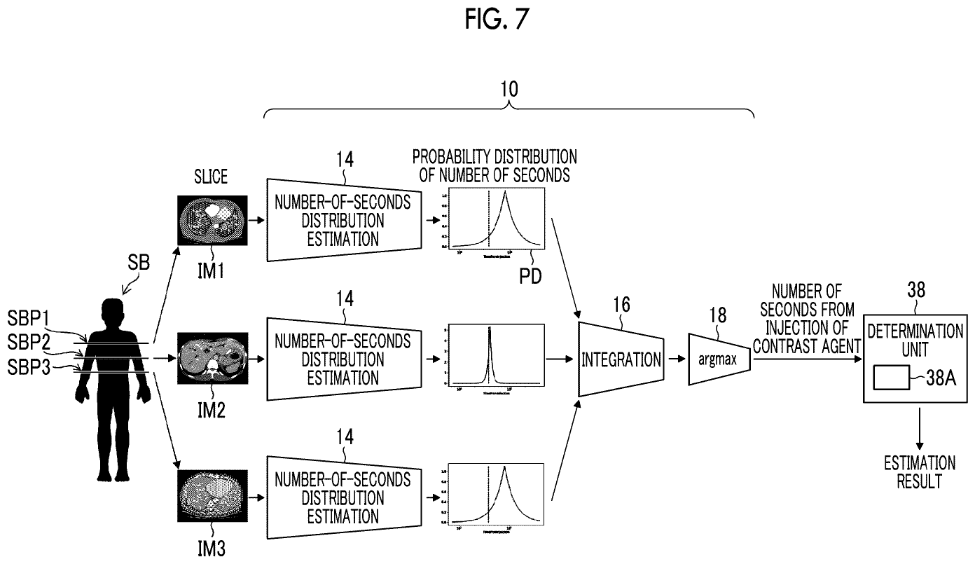

FIG. 7 is a conceptual diagram illustrating an outline of a process by the contrast state determination device 10 according to the third embodiment. Here, an example of the contrast state determination device 10 that estimates a distribution of the number of seconds from the injection of the contrast agent on the basis of a plurality of input slice images and determines the contrast state on the basis of the estimated distribution of the number of seconds will be described.

As illustrated in FIG. 7, the contrast state determination device 10 includes a number-of-seconds distribution estimation unit 14 that receives the input of the images IM and estimates the probability distribution of the number of seconds (hereinafter, referred to as a “number-of-seconds distribution”), an integration unit 16 that integrates a plurality of number-of-seconds distributions PD estimated from a plurality of inputs, a maximum point specification unit 18 that specifies the number of seconds at which the probability is maximized from a new distribution (hereinafter referred to as an “integrated distribution”) obtained by the integration process, and a determination unit 38 that makes the specified number of seconds correspond to the contrast time phase to determine the contrast time phase. The contrast time phase converted by the determination unit 38 is output as the final result.

Further, three number-of-seconds distribution estimation units 14 are illustrated in FIG. 7 in order to illustrate a flow of a process in a case where three different images IM are input. However, the number-of-seconds distribution estimation units 14 to which each slice image IM is input are the same (single) processing unit.

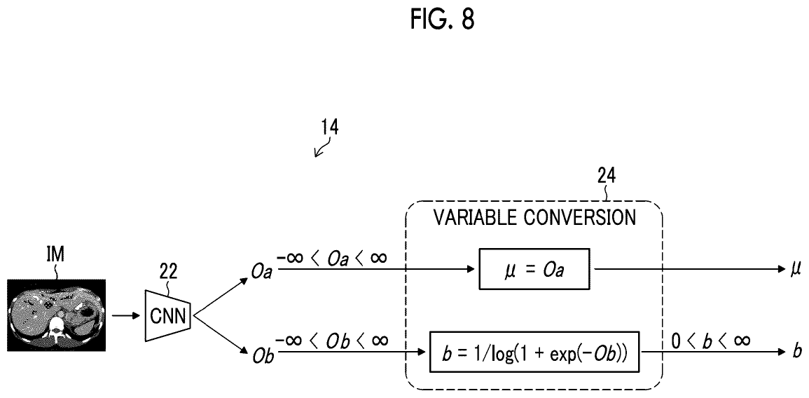

FIG. 8 is a diagram illustrating Example 1 of a process of the number-of-seconds distribution estimation unit 14. The number-of-seconds distribution estimation unit 14 includes a regression estimation unit 22 and a variable conversion unit 24. The regression estimation unit 22 includes a trained model that has been trained by machine learning such that the trained model receives the input of the images IM and outputs an estimated value Oa of the number of seconds and a score value Ob indicating the certainty (certainty factor) of the estimated value Oa. The trained model as a regression model applied to the regression estimation unit 22 is configured using, for example, a convolutional neural network. The numerical range of the estimated value Oa of the number of seconds output from the regression estimation unit 22 may be “−∞<Oa<∞”, and the numerical range of the score value Ob of the certainty may be “−∞<Ob<∞”. In addition, the regression model is not limited to the CNN, and various machine learning models can be applied.

The variable conversion unit 24 performs variable conversion on the estimated value Oa of the number of seconds and the score value Ob of the certainty according to the following Expressions (1) and (2) to generate parameters μ and b of the probability distribution model, respectively.

μ=Oa (1)

b=1/log(1+exp(−Ob)) (2)

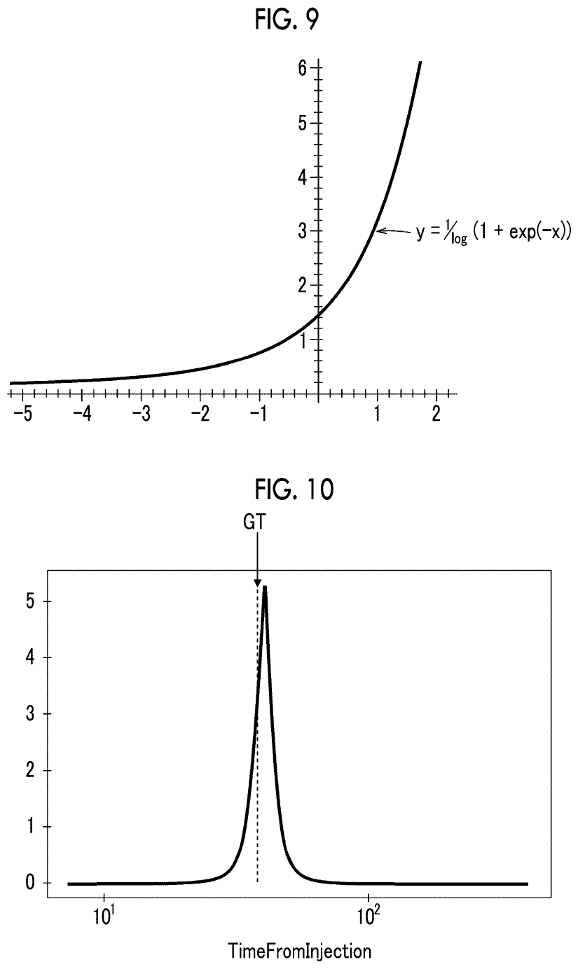

The function represented by Expression (2) is an example of mapping that converts the score value Ob of the certainty into a value b in a positive region. FIG. 9 is a graph of a function y=1/log(1+exp(−x)) used for the variable conversion represented by Expression (2). The parameter u is an example of a “first parameter” according to the present disclosure. The parameter b is an example of a “second parameter” according to the present disclosure.

In the third embodiment, the Laplace distribution is applied as the probability distribution model of the number-of-seconds distribution. The Laplace distribution is represented by a function represented by the following Expression (3).

$$f ( x ; μ , b ) = \frac{1}{2 b} exp ( - \frac{\vert x - μ \vert}{b} ) \qquad ( 3 )$$

The reason for converting the score value Ob of the certainty into the positive value b is related to the application of the Laplace distribution as the probability distribution model of the number-of-seconds distribution. The reason is that, in a case where the parameter b is a negative value (b<0), the Laplace distribution is not established as the probability distribution, and thus it is necessary to ensure that the parameter b is a positive value (b>0).

FIG. 10 illustrates an example of a graph of the number-of-seconds distribution estimated from the parameters μ and b estimated by the number-of-seconds distribution estimation unit 14. In addition, a position indicated by a broken line GT in FIG. 10 corresponds to a correct number of seconds (correct answer number of seconds). Estimating a set of the estimated value Oa and the score value Ob of the certainty from the input images IM substantially corresponds to estimating the number-of-seconds distribution. The estimated value Oa of the number of seconds is an example of a “random variable” according to the present disclosure.

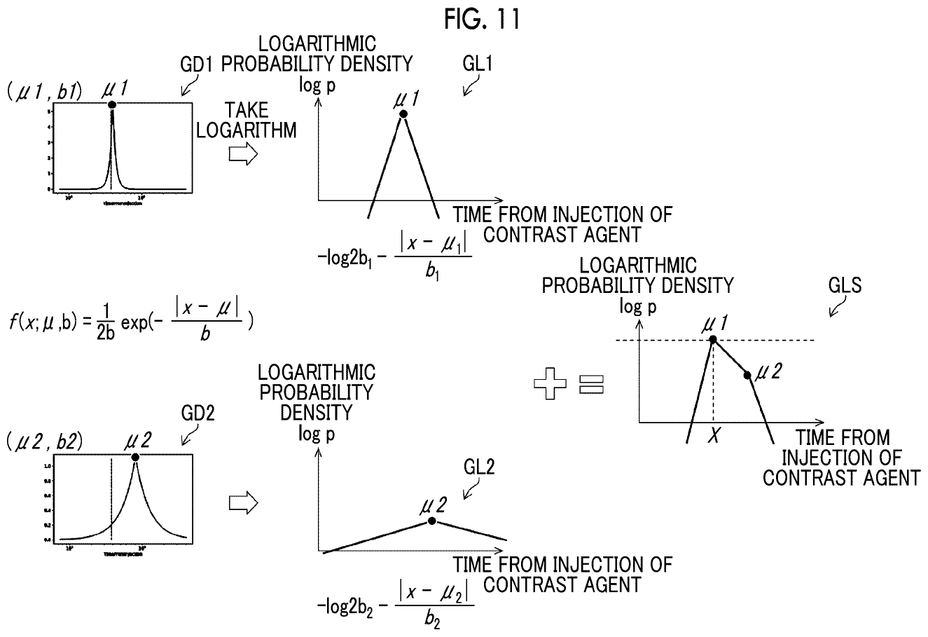

FIG. 11 is a diagram illustrating an example of the processes of the integration unit 16 and the maximum point specification unit 18. Here, for simplicity of description, an example in which two number-of-seconds distributions estimated by the number-of-seconds distribution estimation unit 14 are integrated will be described. However, the same applies to a case where three or more number-of-seconds distributions are integrated.

A graph GD1 illustrated on the upper left side of FIG. 11 is an example of a number-of-seconds distribution (probability distribution P1) represented by parameters μ1 and b1 estimated for the input of the image IM1 (not illustrated in FIG. 11) by the number-of-seconds distribution estimation unit 14. The parameter μ1 is an example of the “first elapsed time”, the parameter b1 is an example of a “first certainty of the first elapsed time”, and the probability distribution P1 is an example of a “first probability distribution model”. The integration unit 16 takes a logarithm of the estimated number-of-seconds distribution to convert the number-of-seconds distribution into a logarithmic probability density and calculates the sum of a plurality of logarithmic probability densities to perform integration. This corresponds to calculating the product of the probabilities at the same number of seconds.

A graph GL1 illustrated in FIG. 11 is an example of a logarithmic probability density log P1 obtained by taking a logarithm of the probability distribution P1. A graph GD2 illustrated on the lower left side of FIG. 11 is an example of a number-of-seconds distribution (probability distribution P2) represented by parameters μ2 and b2 estimated for the input of the image IM2 (not illustrated in FIG. 11) by the number-of-seconds distribution estimation unit 14. The parameter μ2 is an example of the “second elapsed time”, the parameter b2 is an example of a “second certainty of the second elapsed time”, and the probability distribution P2 is an example of a “second probability distribution model”. Further, the parameters μ1 and μ2 are an example of a “plurality of elapsed times”, the parameters b1 and b2 are an example of a “plurality of certainties”, and the probability distributions P1 and P2 are an example of a “plurality of probability distribution models”. A graph GL2 illustrated in FIG. 11 is an example of a logarithmic probability density log P2 obtained by taking a logarithm of the probability distribution P2.

A graph GLS illustrated on the rightmost side of FIG. 11 is an example of a simultaneous logarithmic probability density obtained by integrating the logarithmic probability density log P1 and the logarithmic probability density log P2 and is an example of the “product of the first probability distribution model and the second probability distribution model”. The distribution illustrated in the graph GLS is an example of an “integrated distribution” according to the present disclosure.

The maximum point specification unit 18 specifies a value x of the parameter μ, at which the logarithmic probability is maximized, from the integrated logarithmic probability density. The process of the maximum point specification unit 18 can be represented by the following Expression (4).

$$\begin{matrix}x = {arg max}_{x} {\sum }_{i} ( - log 2 {b}_{i} - \frac{\vert x - {μ}_{i} \vert}{{b}_{i}} ) \\ = {arg max}_{x} {\sum }_{i} ( log {b}_{i} + \frac{\vert x - {μ}_{i} \vert}{{b}_{i}} ) \\ = {arg max}_{x} {\sum }_{i} \frac{\vert x - {μ}_{i} \vert}{{b}_{i}}\end{matrix} \qquad ( 4 )$$

A target function of arg min (a portion after Σ) illustrated on the right side of an equal sign described in the second row of Expression (4) corresponds to a loss function during training in machine learning which will be described below. In addition, the right side of the equal sign described in the third row corresponds to a weighted median expression. A parameter bi corresponding to the weight during integration dynamically changes according to the output of the regression estimation unit 22.

In the case of the integrated logarithmic probability density illustrated in the graph GLS of FIG. 11, the input value (maximum point) at which the simultaneous logarithmic probability is maximized is μ1, and μ1 is selected as the final estimation result (final result). In addition, μ1 is the estimation result of the image IM1 among the plurality of input slice images IMi. In FIG. 11, the number-of-seconds distribution is converted into the logarithmic probability density, and calculation is performed. In short, a process that derives a value, at which the simultaneous probability is maximized, as the final result, considering the simultaneous probability of a plurality of number-of-seconds distributions (probability distributions) estimated from a plurality of different inputs is performed.

The Laplace distribution is adopted as the probability distribution model, and the integrated distribution (simultaneous probability distribution) has the form of a weighted median. Therefore, in a case where some of a plurality of estimation results are values that deviate significantly due to artifacts or the like, it is possible to suppress the influence of the outliers and to obtain an estimated value with high accuracy.

### Example 1 of Machine Learning Method

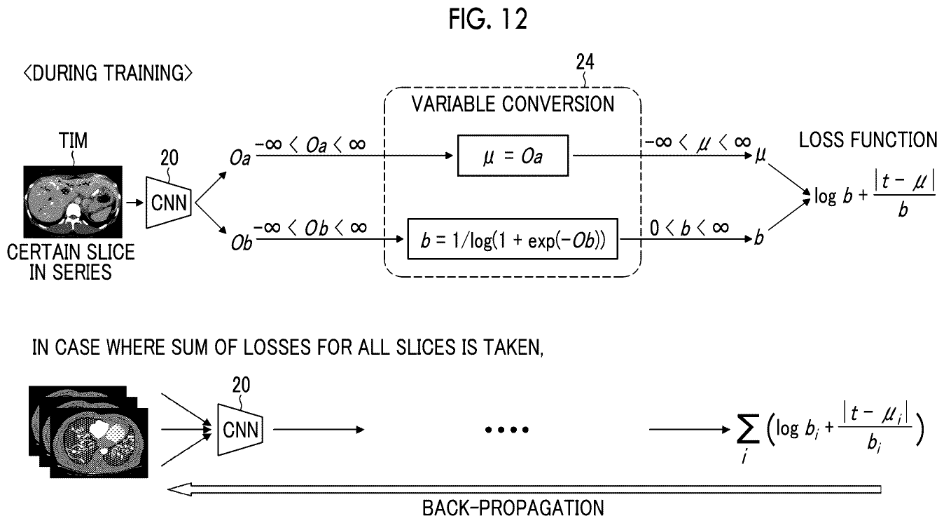

FIG. 12 is a diagram schematically illustrating an example of a machine learning method for generating a regression model applied to the number-of-seconds distribution estimation unit 14. Training data used for machine learning includes an image TIM as data for input and a correct answer data (teaching signal t) corresponding to the input. The image TIM may be a slice image constituting an image series of three-dimensional CT data, and the teaching signal t may be a value indicating the number of seconds (ground truth) from the injection of the contrast agent in a case where the series to which the slice image belongs is captured.

For example, a plurality of training data items are generated by linking the corresponding teaching signals t to all of the slices the image series. The “linking” may be paraphrased as correspondence or association. The term “training” is synonymous with “learning”. The same teaching signal t may be linked to the slices of the same image series. That is, the teaching signal t may be linked in units of image series. For a plurality of image series, similarly, a plurality of training data items are generated by linking the corresponding teaching signals t to the slices. An aggregate of the plurality of training data items generated in this way is used as a training data set.

The learning model 20 (an example of a “second learning model”) is configured using the CNN. The learning model 20 is used in combination with a variable conversion unit 24. In addition, the variable conversion unit 24 may be integrally incorporated into the learning model 20.

In a case where the image TIM read out from the training data set is input to the learning model 20, the learning model 20 outputs the estimated value Oa of the number of seconds and the score value Ob of the certainty of the estimated value Oa. The variable conversion unit 24 performs variable conversion to convert the estimated value Oa and the score value Ob into a parameter μ and a parameter b of the probability distribution model, respectively.

A loss function L used during training is defined by the following Expression (5).

$$L = log b + \frac{\vert t - μ \vert}{b} \qquad ( 5 )$$

As illustrated on the lower side of FIG. 12, in a case where the sum of losses for all of slices of the same image series is taken, the sum is represented by the following Expression (6).

$${\sum }_{i} ( log {b}_{i} + \frac{\vert t - {μ}_{i} \vert}{{b}_{i}} ) \qquad ( 6 )$$

A suffix i is an index for identifying each slice. A back-propagation method is applied using the sum of the losses represented by Expression (6), and the learning model 20 is trained (the parameters of the learning model 20 are updated) using a stochastic gradient descent method in the same manner as in normal CNN training. The sum of the losses calculated by Expression (6) is an example of a “calculation result of a loss function” according to the present disclosure. The learning model 20 is trained using a plurality of training data items including a plurality of image series such that the parameters of the learning model 20 are optimized to obtain a trained model. The trained model obtained in this way is applied as the regression model of the number-of-seconds distribution estimation unit 14.

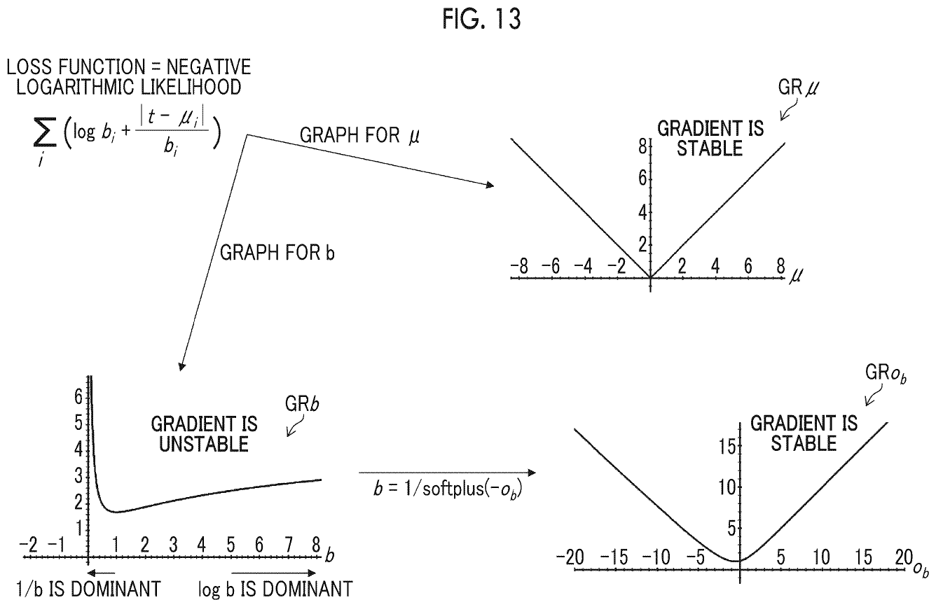

FIG. 13 is a diagram illustrating the loss function used during training. The loss function is negative logarithmic likelihood and directly optimizes an expression that is used for regression estimation using learning. The logarithmic likelihood of the teaching signal t at the number of seconds is optimized by learning. A graph for the parameter μ of the loss function represented by Expression (5) is a graph GRμ in FIG. 13. In the graph GRμ, the gradient with respect to the parameter μ is stable.

On the other hand, a graph for the parameter b of the loss function represented by Expression (5) is a graph GRb in FIG. 13. In the graph GRb, the gradient with respect to the parameter b is unstable. 1/b is dominant in a region in which the value of b is small, and log b is dominant in a region in which the value of b is large.

The graph GRb in which the gradient is unstable is converted into a graph GROb by performing variable conversion to convert the parameter b using a function such as b=1/softplus(−Ob). A softplus function is defined as softplus(x)=log(1+exp(x)). The function used for the variable conversion of the parameter b is a function that approaches −1/x at x→−∞ and approaches exp(x) at x→∞. The use of this function makes it possible to cancel the instability of the gradient.

The machine learning method of the learning model 20 described with reference to FIGS. 12 and 13 is an example of a “method for generating a trained model” according to the present disclosure.

### <<Functional Configuration of Contrast State Determination Device 10>>

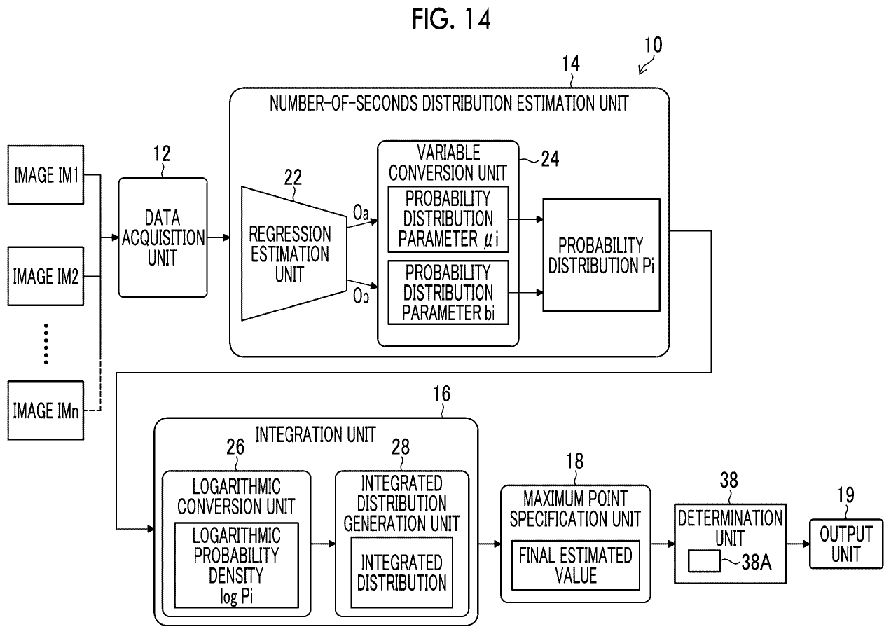

FIG. 14 is a functional block diagram illustrating an outline of processing functions of the contrast state determination device 10 according to the third embodiment. The processor 102 of the contrast state determination device 10 executes the contrast state determination program 130 stored in the memory 104A to function as the data acquisition unit 12, the number-of-seconds distribution estimation unit 14, the integration unit 16, the maximum point specification unit 18, the determination unit 38, and the output unit 19.

The data acquisition unit 12 receives the input of data to be processed. In the example illustrated in FIG. 14, the data acquisition unit 12 acquires the images IMi which are the slice images sampled from CT data. A subscript i (where i is 1 to n) indicates an index number for identifying a plurality of images. FIG. 14 illustrates that n different images can be input. n may be an integer equal to or greater than 2. The data acquisition unit 12 may perform a process of cutting out slice images from the CT data at equal intervals or may acquire the slice images sampled in advance using a processing unit (not illustrated) or the like.

The images IMi acquired through the data acquisition unit 12 are input to the regression estimation unit 22 of the number-of-seconds distribution estimation unit 14. The regression estimation unit 22 outputs a set of the estimated value Oa of the number of seconds and the score value Ob indicating the certainty of the estimated value Oa from each of the input images IMi.

The variable conversion unit 24 converts the estimated value Oa output from the regression estimation unit 22 into a parameter μi of the probability distribution model. The variable conversion unit 24 converts the score value Ob of the certainty output from the regression estimation unit 22 into a parameter bi of the probability distribution model. A probability distribution Pi of the number of seconds is estimated by these two parameters μi and bi.

A plurality of images IMi (i=1 to n) in the same series are input, and a set of the estimated value Oa and the score value Ob is estimated for each of the images IMi and is converted into a set of the parameters μi and bi. Then, the probability distribution Pi of the number of seconds is estimated. A plurality of sets of the estimated value Oa and the score value Ob estimated from each of the images IMi are an example of “a plurality of sets of estimation results” according to the present disclosure.

The integration unit 16 performs a process of integrating a plurality of probability distributions Pi obtained on the basis of the input of the plurality of images IMi. In FIG. 14, a logarithmic conversion unit 26 takes a logarithm of the probability distribution Pi to convert the probability distribution Pi into a logarithmic probability density log Pi, and an integrated distribution generation unit 28 calculates the sum of the logarithmic probability densities log Pi to obtain an integrated distribution.

The maximum point specification unit 18 specifies the value of the number of seconds (maximum point), at which the probability is maximized, from the integrated distribution and outputs the specified value of the number of seconds. In addition, the maximum point specification unit 18 may be incorporated into the integration unit 16.

The determination unit 38 determines the contrast state from the value of the number of seconds specified by the maximum point specification unit 18. The determination unit 38 includes a conversion table 38A. The conversion table 38A is stored in the computer-readable medium 104. In the conversion table 38A, the elapsed time from the start of the injection of the contrast agent and the contrast time phase are associated with each other. For example, in the case of liver contrast imaging, in the conversion table 38A, less than 50 seconds from the start of the injection of the contrast agent is associated with the arterial phase, 50 seconds or more and less than 120 seconds is associated with the portal phase, and 120 seconds or more is associated with the equilibrium phase. The determination unit 38 determines the contrast state using the conversion table 38A.

In a case where the number of seconds is estimated from a plurality of images and the average thereof is used as the final result, the result may deteriorate due to the number of seconds output from the image which is unsuitable for estimation. According to the third embodiment, a certainty factor is calculated together with the number of seconds, and weighting is performed according to the certainty factor. Therefore, the result is less likely to be affected by the estimated outlier and is robust.

### Fourth Embodiment

In the third embodiment, the Laplace distribution is used as the probability distribution model of the number-of-seconds distribution. However, the present invention is not limited thereto, and other probability distribution models may be applied. In the fourth embodiment, an example will be described in which a Gaussian distribution is used instead of the Laplace distribution. The fourth embodiment is different from the third embodiment in the content of the processes of the processing units of the number-of-seconds distribution estimation unit 14, the integration unit 16, and the maximum point specification unit 18.

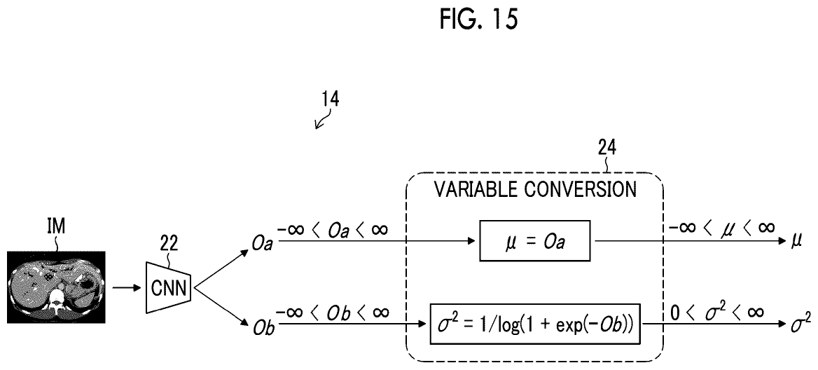

FIG. 15 is a diagram illustrating Example 2 of the process of the number-of-seconds distribution estimation unit 14 in the contrast state determination device 10 according to the fourth embodiment. A process illustrated in FIG. 15 is applied instead of the process described with reference to FIG. 8.

The variable conversion unit 24 according to the fourth embodiment converts the score value Ob of the certainty into a parameter σ² using the following Expression (7) instead of Expression (2).

σ²=1/log(1+exp(−Ob)) (7)

σ² plays a role of certainty. σ² corresponds to a dispersion, and σ corresponds to a standard deviation.

The Gaussian distribution is represented by a function represented by the following Expression (8).

$$f ( x ; μ , σ ) = \frac{1}{\sqrt{2 π {σ}^{2}}} exp ( - \frac{{( x - μ )}^{2}}{2 {σ}^{2}} ) \qquad ( 8 )$$

The reason for converting the score value Ob into a positive value (σ²) is the same as that in the third embodiment. The reason is that, in a case where the parameter σ² is a negative value, the Gaussian distribution is not established as the probability distribution and it is necessary to ensure that the parameter σ² is a positive value (σ²>0).

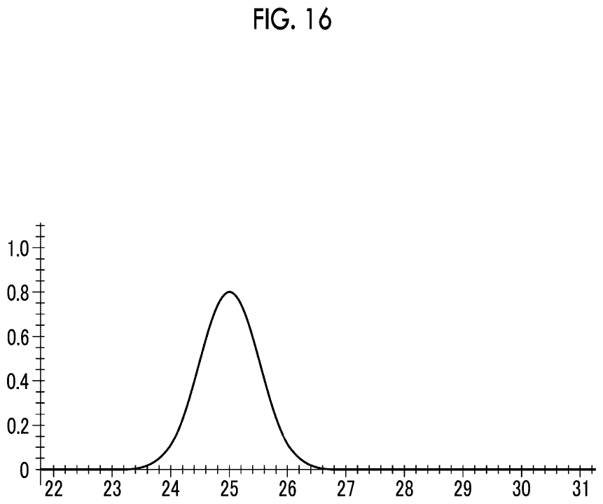

FIG. 16 illustrates an example of a graph of the number-of-seconds distribution that is estimated on the basis of the parameters μ and σ² estimated by the number-of-seconds distribution estimation unit 14.

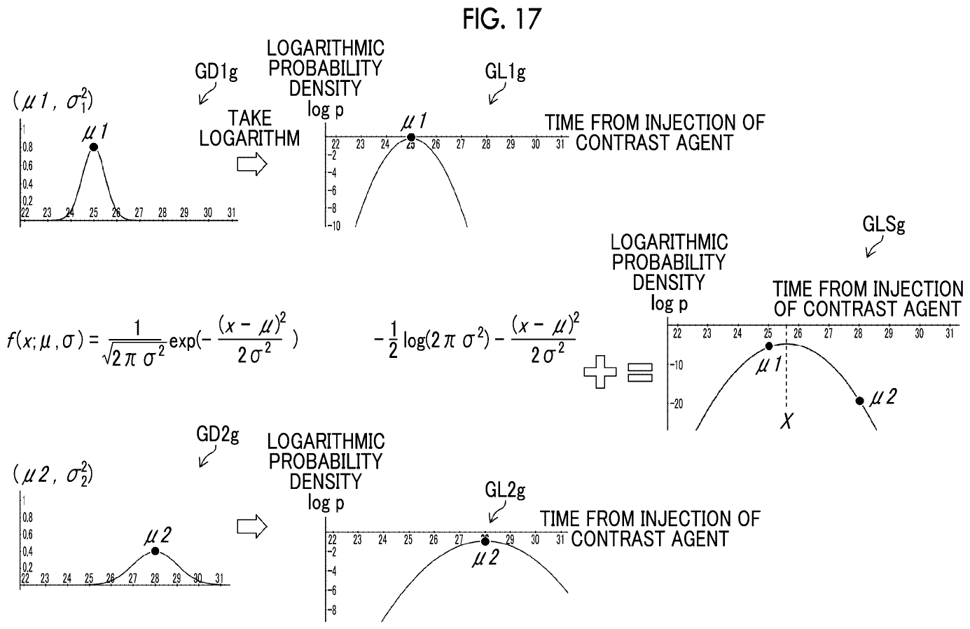

FIG. 17 is a diagram illustrating an example of processes of the integration unit 16 and the maximum point specification unit 18 of the contrast state determination device 10 according to the fourth embodiment. Here, an example in which two number-of-seconds distributions estimated by the number-of-seconds distribution estimation unit 14 are integrated will be described.

A graph GD1g illustrated on the upper left side of FIG. 17 is an example of the number-of-seconds distribution (probability distribution P1) represented by parameters μ1 and σ²₁ estimated by the number-of-seconds distribution estimation unit 14 illustrated in FIG. 15. The integration unit 16 takes a logarithm of the estimated number-of-seconds distribution to convert the number-of-seconds distribution into a logarithmic probability density and calculates the sum of a plurality of logarithmic probability densities to perform integration. This corresponds to calculating the product of the probabilities at the same number of seconds.

A graph GL1g illustrated in FIG. 17 is an example of a logarithmic probability density log P1 obtained by taking a logarithm of the probability distribution P1. A graph GD2g illustrated on the lower left side of FIG. 17 is an example of the number-of-seconds distribution (probability distribution P2) represented by parameters μ2 and σ₂² estimated by the number-of-seconds distribution estimation unit 14. A graph GL2g illustrated in FIG. 17 is an example of a logarithmic probability density log P2 obtained by taking a logarithm of the probability distribution P2.

A graph GLSg illustrated on the rightmost side of FIG. 17 is an example of a simultaneous logarithmic probability density obtained by integrating the logarithmic probability density log P1 and the logarithmic probability density log P2.

The maximum point specification unit 18 specifies a value x, at which the logarithmic probability is maximized, from the integrated simultaneous logarithmic probability density. The process of the maximum point specification unit 18 can be represented by the following Expression (9).

$$\begin{matrix}x = {arg max}_{x} {\sum }_{i} ( - log 2 π {σ}_{i}^{2} - \frac{{( x - {μ}_{i} )}^{2}}{2 {σ}^{2}} ) \\ = {arg max}_{x} {\sum }_{i} ( log {σ}_{i}^{2} + \frac{{( x - {μ}_{i} )}^{2}}{2 {σ}^{2}} ) \\ = {arg max}_{x} {\sum }_{i} \frac{{( x - {μ}_{i} )}^{2}}{{σ}^{2}}\end{matrix} \qquad ( 9 )$$

A target function of arg min (a portion after Σ) illustrated on the right side of an equal sign described in the second row of Expression (9) corresponds to a loss function during training in machine learning which will be described below. In addition, the right side of the equal sign described in the third row corresponds to a weighted average expression.

In the case of the integrated logarithmic probability density in the graph GLSg illustrated in FIG. 17, the input value x (maximum point) at which the logarithmic probability is maximized is selected as the final estimation result (final result).

### Example 2 of Machine Learning Method

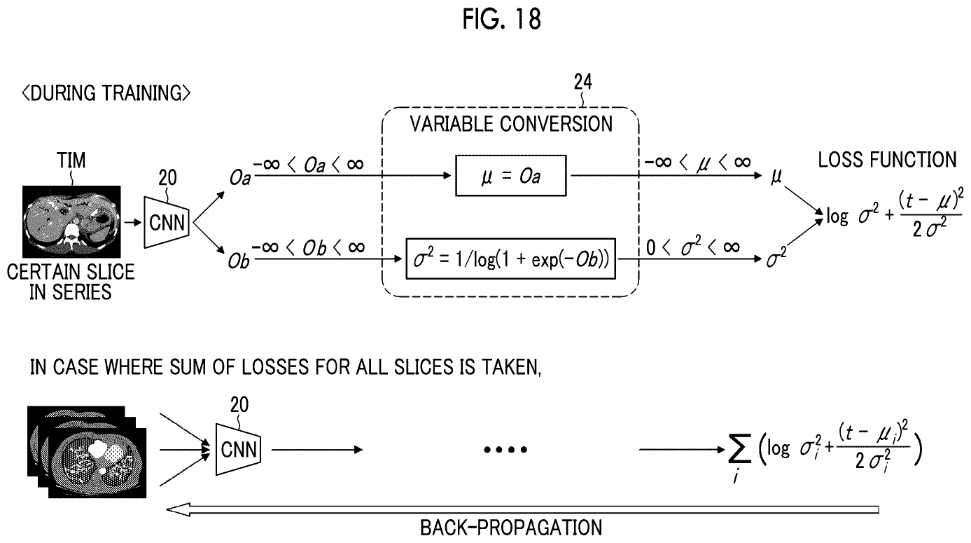

FIG. 18 is a diagram schematically illustrating an example of a machine learning method for generating a regression model applied to the number-of-seconds distribution estimation unit 14 according to the fourth embodiment. Training data used for learning may be the same as that in the third embodiment. Differences from FIG. 12 will be described with reference to FIG. 18.

In a case where the image TIM read out from the training data set is input to the learning model 20, the learning model 20 outputs the estimated value Oa of the number of seconds and the score value Ob of the certainty of the estimated value Oa. The variable conversion unit 24 performs variable conversion to convert the estimated value Oa and the score value Ob of the certainty into parameters μ and σ² of the probability distribution model, respectively.

A loss function L during training is defined by the following Expression (10).

$$L = log {σ}^{2} + \frac{{( t - μ )}^{2}}{2 {σ}^{2}} \qquad ( 10 )$$

As illustrated on the lower side of FIG. 18, in a case where the sum of losses is taken for all of slices of the same image series, the sum is represented by the following Expression (11).

$${\sum }_{i} ( log {σ}_{1}^{2} + \frac{{( t - {μ}_{i} )}^{2}}{2 {σ}_{i}^{2}} ) \qquad ( 11 )$$

The back-propagation method is applied using the sum of the losses represented by Expression (11), and the learning model 20 is trained using the stochastic gradient descent method in the same manner as in normal CNN training. The learning model 20 is trained using a plurality of training data items including a plurality of image series such that the parameters of the learning model 20 are optimized to obtain a trained model. The trained model obtained in this way is applied to the number-of-seconds distribution estimation unit 14.

### Modification Example 1

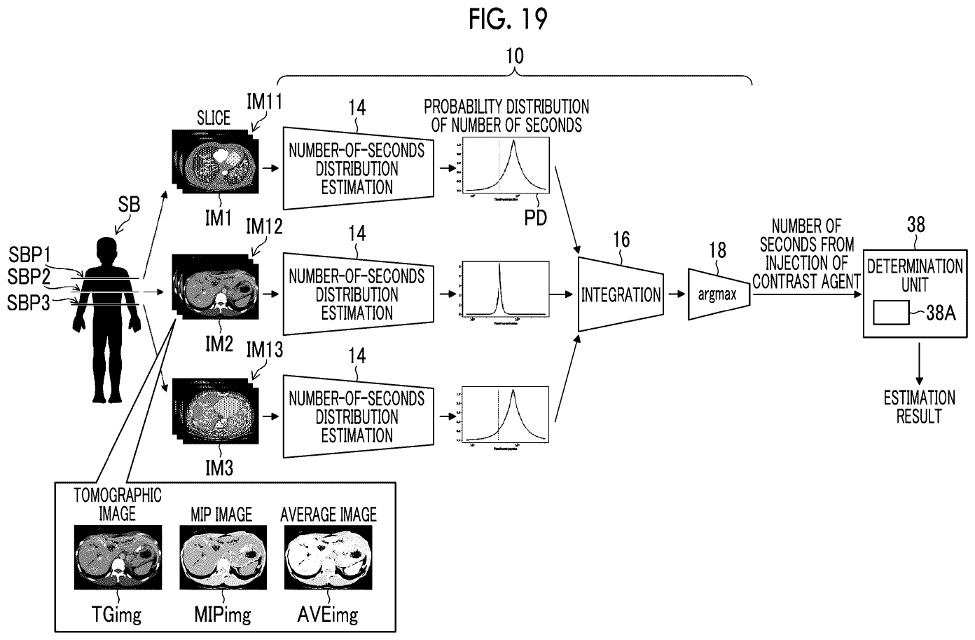

In the first to fourth embodiments, the images IM1, IM2, and IM3 which are the slice images (tomographic images) obtained by dividing three-dimensional CT data into slices at equal intervals are used as the input. However, the image to be processed is not limited thereto. For example, as illustrated in FIG. 19, the images to be processed may be a two-dimensional image IM11 (an example of the “first two-dimensional image”) including information of the image IM1 (an example of the “first slice image”), a two-dimensional image IM12 (an example of the “second two-dimensional image”) including information of the image IM2 (an example of the “second slice image”), and a two-dimensional image IM13 including information of the image IM3. The two-dimensional image may be a tomographic image TGimg, a maximum intensity projection (MIP) image MIPimg configured at equal intervals, an average image AVEimg generated from a plurality of slice images, or the like.

### Modification Example 2

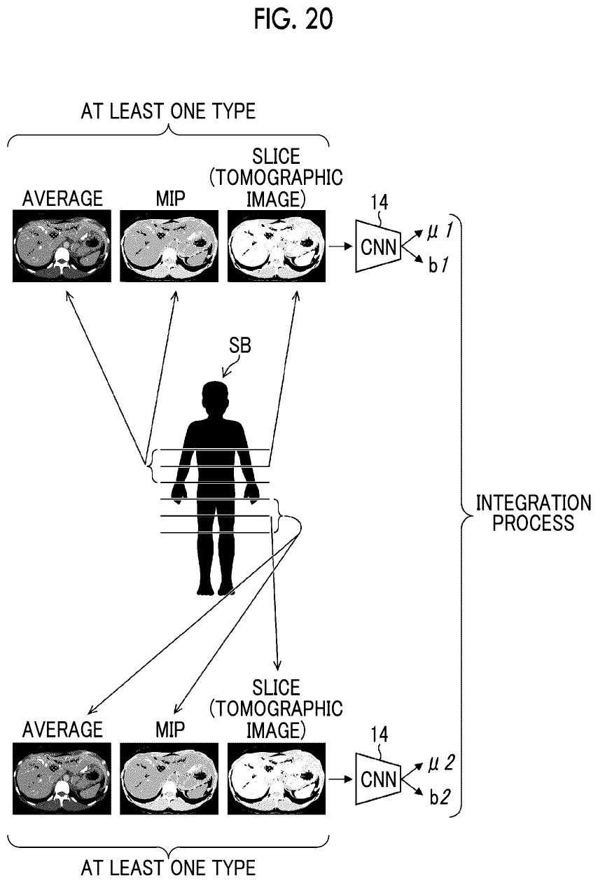

The input to the time phase estimation unit 30, the number-of-seconds estimation unit 34, and the number-of-seconds distribution estimation unit 14 may be a combination of a plurality of types of data elements. For example, as illustrated in FIG. 20, at least one of the slice image, the MIP image, or the average image which is a partial image of CT data of the same series can be used as the input. A combination of the plurality of types of images may be input to the time phase estimation unit 30, the number-of-seconds estimation unit 34, and the number-of-seconds distribution estimation unit 14. For example, a combination of the average image and the MIP image may be input to the number-of-seconds distribution estimation unit 14 to estimate the number-of-seconds distribution. The MIP image and the average image are examples of a generated image that is generated from partial images of three-dimensional CT data.

### <<Example of Configuration of Medical Information System>>

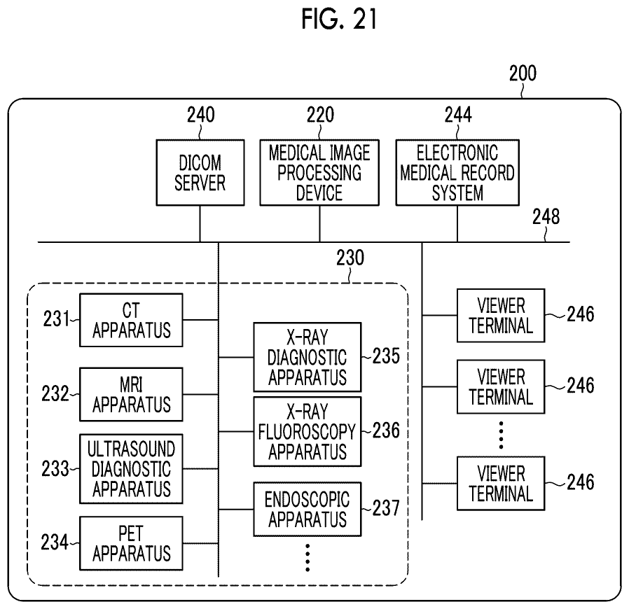

FIG. 21 is a block diagram illustrating an example of a configuration of a medical information system 200 including a medical image processing device 220. The contrast state determination device 10 described in the first to fourth embodiments is incorporated into, for example, the medical image processing device 220. A medical information system 200 is a computer network constructed in a medical institution such as a hospital. The medical information system 200 includes a modality 230 that captures a medical image, a DICOM server 240, the medical image processing device 220, an electronic medical record system 244, and a viewer terminal 246. These elements are connected via a communication line 248. The communication line 248 may be a local communication line in the medical institution. Further, a portion of the communication line 248 may be a wide area communication line.

Specific examples of the modality 230 include a CT apparatus 231, a magnetic resonance imaging (MRI) apparatus 232, an ultrasound diagnostic apparatus 233, a positron emission tomography (PET) apparatus 234, an X-ray diagnostic apparatus 235, an X-ray fluoroscopy apparatus 236, and an endoscopic apparatus 237. There may be various combinations of types of the modalities 230 connected to the communication line 248 for each medical institution.

The DICOM server 240 is a server that operates according to the specifications of DICOM. The DICOM server 240 is a computer that stores various types of data including the images captured by the modality 230 and that manages various types of data. The DICOM server 240 comprises a large-capacity external storage device and a database management program. The DICOM server 240 communicates with other devices through the communication line 248 to transmit and receive various types of data including image data. The DICOM server 240 receives the image data generated by the modality 230 and other various types of data through the communication line 248, stores the data in a recording medium, such as a large-capacity external storage device, and manages the data. In addition, the storage format of the image data and the communication between the devices via the communication line 248 are based on a DICOM protocol.

The medical image processing device 220 can acquire data from the DICOM server 240 or the like via the communication line 248. The medical image processing device 220 performs image analysis and various other types of processes on the medical images captured by the modality 230. The medical image processing device 220 may be configured to perform, for example, various analysis processes, such as computer aided diagnosis and computer aided detection (CAD), including a process of recognizing a lesion region or the like from an image, a process of specifying a classification, such as a disease name, and a segmentation process of recognizing a region of an organ or the like, in addition to the processing functions of the contrast state determination device 10. Further, the medical image processing device 220 can transmit a processing result to the DICOM server 240 and the viewer terminal 246. Furthermore, the processing functions of the medical image processing device 220 may be provided in the DICOM server 240 or the viewer terminal 246.

The various types of data stored in a database of the DICOM server 240 and various types of information including processing results generated by the medical image processing device 220 can be displayed on the viewer terminal 246.

The viewer terminal 246 is a terminal for image viewing called a picture archiving and communication systems (PACS) viewer or a DICOM viewer. A plurality of viewer terminals 246 may be connected to the communication line 248. The form of the viewer terminal 246 is not particularly limited and may be, for example, a personal computer, a workstation, or a tablet terminal.

### <<For Program for Operating Computer>>

A program that causes a computer to implement the processing functions of the contrast state determination device 10 can be recorded on a computer-readable medium which is a non-transitory tangible information storage medium, such as an optical disk, a magnetic disk, or a semiconductor memory. Then, the program can be provided through the information storage medium.

In addition, instead of the aspect in which the program is stored in the non-transitory tangible computer-readable medium and then provided, program signals may be provided as a download service using a telecommunication line such as the Internet.

Furthermore, some or all of the processing functions of the contrast state determination device 10 may be implemented by cloud computing or may be provided as a Software as a Service (SasS) service.

### <<For Hardware Configuration of Each Processing Unit>>

A hardware structure of processing units performing various processes, such as the data acquisition unit 12, the number-of-seconds distribution estimation unit 14, the integration unit 16, the maximum point specification unit 18, the output unit 19, the regression estimation unit 22, the variable conversion unit 24, the logarithmic conversion unit 26, the integrated distribution generation unit 28, the time phase estimation unit 30, and the integration unit 32, in the contrast state determination device 10 is, for example, the following various processors.

The various processors include, for example, a CPU which is a general-purpose processor executing a program to function as various processing units, a GPU which is a processor specializing in image processing, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform a specific process.

One processing unit may be configured by one of the various processors or a combination of two or more processors of the same type or different types. For example, one processing unit may be configured by a plurality of FPGAs, a combination of a CPU and an FPGA, or a combination of a CPU and a GPU. Further, a plurality of processing units may be configured by one processor. A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As described above, various processing units are configured using one or more of the various processors as a hardware structure.

In addition, specifically, the hardware structure of the various processors is an electric circuit (circuitry) obtained by combining circuit elements such as semiconductor elements.

### <<Others>>

The present disclosure is not limited to the above-described embodiments, and various modifications can be made without departing from the gist of the technical idea of the present disclosure.

### EXPLANATION OF REFERENCES

10: contrast state determination device
12: data acquisition unit
14: number-of-seconds distribution estimation unit
16: integration unit
18: maximum point specification unit
19: output unit
20: learning model
22: regression estimation unit
24: variable conversion unit
26: logarithmic conversion unit
28: integrated distribution generation unit
30: time phase estimation unit
30A: trained model
32: integration unit
34: number-of-seconds estimation unit
34A: trained model
36: integration unit
38: determination unit
38A: conversion table
102: processor
104: computer-readable medium
104A: memory
104B: storage
106: communication interface
108: input/output interface
110: bus
114: input device
116: display device
130: contrast state determination program
200: medical information system
220: medical image processing device
230: modality
231: CT apparatus
232: MRI apparatus
233: Ultrasound diagnostic apparatus
234: PET apparatus
235: X-ray diagnostic apparatus
236: X-ray fluoroscopy apparatus
237: endoscopic apparatus
240: DICOM server
244: electronic medical record system
246: viewer terminal
248: communication line
GD1: graph
GD1g: graph
GD2: graph
GD2g: graph
GL1: graph
GL1g: graph
GL2: graph
GL2g: graph
GLS: graph
GLSg: graph
GRb: graph
GRμ: graph
GROb: graph
IM: image
IM1, IM2, IM3, IMi, IMn: image
TIM: image
IM11, IM12, IM13: two-dimensional image
Oa: estimated value
Ob: score value
P1, P2, Pi: probability distribution
PD: number-of-seconds distribution
PS1, PS2, PS3, PS11, PS12, PS13, PS14: estimation result
SB: patient
SBP1, SBP2, SBP3: position
S1 to S5: step of contrast state determination method

What is claimed is:

1. A contrast state determination device comprising:
at least one processor; and
at least one memory that stores commands to be executed by the at least one processor,
wherein the at least one processor
acquires a plurality of two-dimensional images including information of slice images of a subject at different positions from a first image series captured before or after a contrast agent is injected into the subject,
estimates an index value related to a contrast state from each of the plurality of two-dimensional images, and
determines the contrast state of the first image series on the basis of each of a plurality of the estimated index values.

2. The contrast state determination device according to claim 1,
wherein the index value is a certainty of belonging to each of a plurality of the contrast states, and
the at least one processor derives an index value obtained by integrating the plurality of index values and determines the contrast state of the first image series on the basis of the integrated index value.

3. The contrast state determination device according to claim 2, further comprising:
a first learning model that, in a case where a two-dimensional image based on an image series captured before or after the contrast agent is injected into the subject is input, outputs a certainty of belonging to each of the plurality of contrast states,
wherein the at least one processor inputs the plurality of two-dimensional images to the first learning model to estimate the plurality of index values.

4. The contrast state determination device according to claim 1,
wherein the index value is an elapsed time from the injection of the contrast agent into the subject, and
the at least one processor derives an elapsed time obtained by integrating a plurality of the elapsed times which are the plurality of index values and determines the contrast state of the first image series on the basis of the integrated elapsed time.

5. The contrast state determination device according to claim 1,
wherein the index values are an elapsed time from the injection of the contrast agent into the subject and a certainty of the elapsed time, and
the at least one processor derives an elapsed time obtained by integrating a plurality of the elapsed times which are the plurality of index values on the basis of a plurality of the certainties which are the plurality of index values and determines the contrast state of the first image series on the basis of the integrated elapsed time.

6. The contrast state determination device according to claim 5,
wherein the at least one processor derives the integrated elapsed time on the basis of a product of a plurality of probability distribution models having the elapsed time and the certainty as parameters.

7. The contrast state determination device according to claim 5, further comprising:
a second learning model that, in a case where a two-dimensional image based on an image series captured before or after the contrast agent is injected into the subject is input, outputs the elapsed time from the injection of the contrast agent into the subject and the certainty of the elapsed time,
wherein the at least one processor inputs the plurality of two-dimensional images to the second learning model to estimate the plurality of elapsed times and the plurality of certainties.

8. The contrast state determination device according to claim 4, further comprising:
a conversion table in which the elapsed time from the injection of the contrast agent into the subject is associated with the contrast state,
wherein the at least one processor determines the contrast state of the first image series on the basis of the integrated elapsed time and the conversion table.

9. The contrast state determination device according to claim 1, further comprising:
a conversion table in which an elapsed time from the injection of the contrast agent into the subject is associated with the contrast state,
wherein the at least one processor
estimates a plurality of the elapsed times from the injection of the contrast agent into the subject as the plurality of index values,
determines a plurality of the contrast states of the first image series on the basis of the plurality of elapsed times and the conversion table, and
determines the contrast state of the first image series on the basis of the plurality of contrast states.

10. The contrast state determination device according to claim 1,
wherein the two-dimensional image is at least one of the slice image, a maximum intensity projection (MIP) image of a plurality of the slice images, or an average image of the plurality of slice images.

11. The contrast state determination device according to claim 1,
wherein the first image series is a three-dimensional image including a liver of the subject, and
the contrast state includes at least one of a non-contrast phase, an arterial phase, a portal phase, or an equilibrium phase.

12. The contrast state determination device according to claim 1,
wherein the first image series is a three-dimensional image including a kidney of the subject, and
the contrast state includes at least one of a non-contrast phase, a corticomedullary phase, a parenchymal phase, or an excretory phase.

13. A contrast state determination method comprising:
a step of acquiring a plurality of two-dimensional images including information of slice images of a subject at different positions from a first image series captured before or after a contrast agent is injected into the subject;
a step of estimating an index value related to a contrast state from each of the plurality of two-dimensional images; and
a step of determining the contrast state of the first image series on the basis of each of a plurality of the estimated index values.

14. A non-transitory, computer-readable tangible recording medium on which a program for causing, when read by a computer, the computer to execute the contrast state determination method according to claim 13 is recorded.
